(19) United States
(12) Patent Application Publication
(10) Pub. No.: US 2003/0157496 A1
(43) Pub. Date: Aug. 21, 2003

(54) Method for processing biological analysis data and expert biological analysis system therefor

(76) Inventors: Patrick Rambaud, Fontaine Le Port (FR); Lionel Chapy, Chanonat (FR)

Correspondence Address: YOUNG & THOMPSON, 745 SOUTH 23RD STREET 2ND FLOOR, ARLINGTON, VA, 22202

(21) Appl. No.: 10/111,900

(22) PCT Filed: Jan. 18, 2002
(86) PCT No.: PCT/FR02/00218

(51) Int. Cl. C12Q 1/68; G06F 19/00; G01N 33/48; G01N 33/50

(52) U.S. Cl. 435/6; 702/20

(57) ABSTRACT

19 Method of processing biological analysis data, comprising processing of a set of data relating to the biological profile of a human or animal subject, in the form of biological items to which a set of rules supplying findings in the form of statements in natural language are applied. The method also comprises at least an operation pooling a set of findings that have resulted from a group of rules, so as to limit the number of findings which are entered a following group of rules. This method also comprises processing of genetic data relating to this patient in the form of a set of genetic items associated with a set of genes studied in this patient, this treatment comprising the application of rules of genetic interpretation applied at the same time to biological items and to genetic items.

The present invention relates to a method of processing biological analysis data. It also relates to an expert system of biological analysis.

In the field of biological analysis, there are already methods of determining the biological profile of a person from a set of measurements of characteristic physiological parameters. Starting from these biological profiles and data relating to the person concerned such as his age, his sex, his physical condition, the practitioner can then produce a set of findings leading to a diagnosis. A biological profile is comprised in practice of a set of specific profiles, such as a protein profile or lymphocyte typing.

The increase in the number of parameters involved in the determination of a biological profile makes it more and more difficult to establish consistent findings. In order to satisfy the expectations of practitioners who prescribe biological analyses, expert systems of biological analysis have been developed. These expert systems procure for users the processing of a set of items corresponding to biological measurements data and to personal data, and provide findings which can be used directly by the prescribing practitioner.

The methods of processing biological data that are used in these expert systems require a set of rules each applied to a determined combination of items among a global set of items corresponding to a set of measurements, examinations, dosages carried out on a patient or personal data. These rules lead to a set of findings which are drafted beforehand by one or more expert practitioners.

It has been shown in practice that the number of possible theoretical findings in an expert system of biological analysis, intended to integrate as complete as possible a biological profile in the current state of the techniques available in biological analysis, is so high that the feasibility of such an expert system and its implementation on conventional data-processing equipment other than large-capacity calculation and storage machines could be implicated.

The aim of the present invention is to propose a method of processing biological analysis data which on the one hand resolves effectively the question of the volume of data to be processed and consequently render such an expert system realisable, and which on the other hand procures for the practitioner using it a better relevance of the findings for interpretation of the analysis results.

This aim is achieved with a method of processing biological analysis data which comprises processing of a set of data relating to the biological profile of a human or animal subject, in the form of biological items to which are applied a set of rules supplying findings in the form of statements in natural language.

This method is characterised in that the set of rules comprises a plurality of rules each associated to an analysis group among a plurality of analysis groups constituting the biological profile and in that it further comprises at least an operation pooling a set of findings that have resulted from a group of rules, so as to limit the number of findings which are entered a following group of rules.

Such pooling operations have the effect of making possible the implementation of an expert system of biological analysis on a personal office-based or portable data-processing apparatus, without affecting the accuracy and rigour of the findings supplied to the user.

It is to be noted that in the present invention pooling operations relates to pooling operations of findings and not to the fusion of chained rules as taught in U.S. Pat. No. 5,442,792 document which discloses a compiling method for an expert system.

Another aim of the method of data processing according to the invention is to permit the realisation of an expert system of biological analysis which integrates genetic profile data, knowledge of which is henceforth regarded as essential for the diagnosis and treatment of an increasing number of affections and pathologies.

This further aim is achieved with a method of processing data according to the invention characterised in that it also comprises a processing of genetic data relating to this patient in the form of a set of genetic items associated with a set of genes studied in this patient, this processing comprising the application of rules of genetic interpretation applied at the same time to biological items and to genetic items.

With this method combining interpretation of a biological profile and interpretation of a genetic profile, it becomes possible to propose more accurate and more relevant findings due to the taking into account of affections linked with the genes.

It is to be noted that document WO 01/16860 discloses artificial intelligence system for a genetic analysis, which does not involve a pooling operation of a set of findings that have resulted from a group of rules, as proposed by the method according to the invention.

According to another aspect of the invention, an expert system of biological analysis is proposed applying the method of data processing according to the invention.

Other advantages and characteristics of the invention will appear upon examination of the detailed description of an embodiment, which is no way limitative, and of the attached drawings in which:

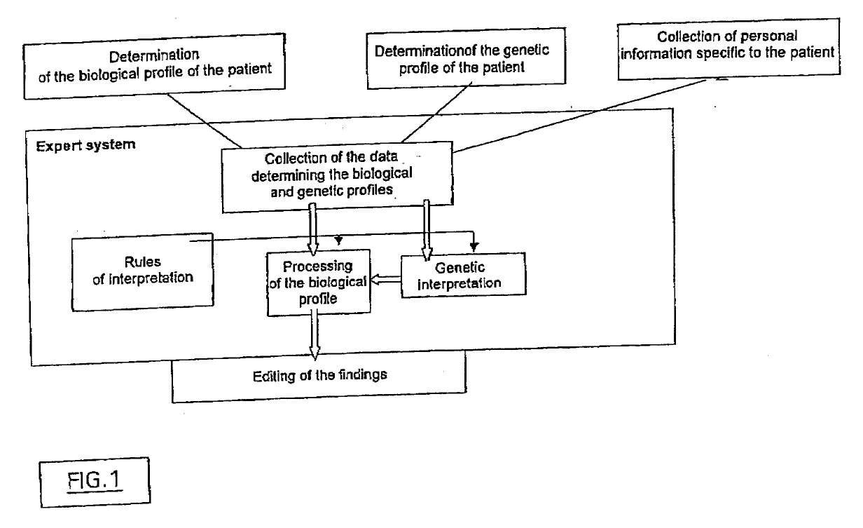

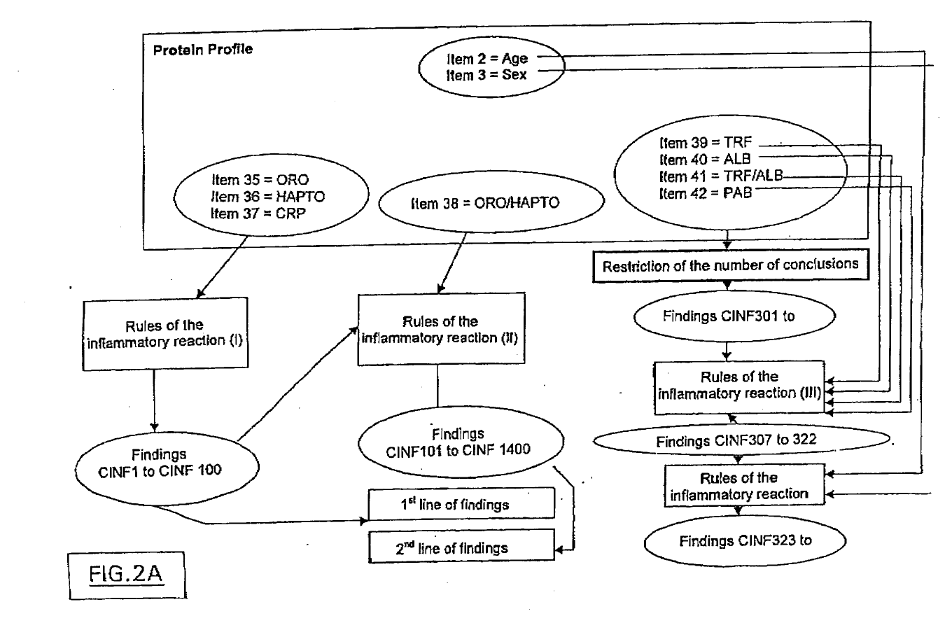

FIG. 1 is a functional diagram of an expert system of biological analysis according to the invention,

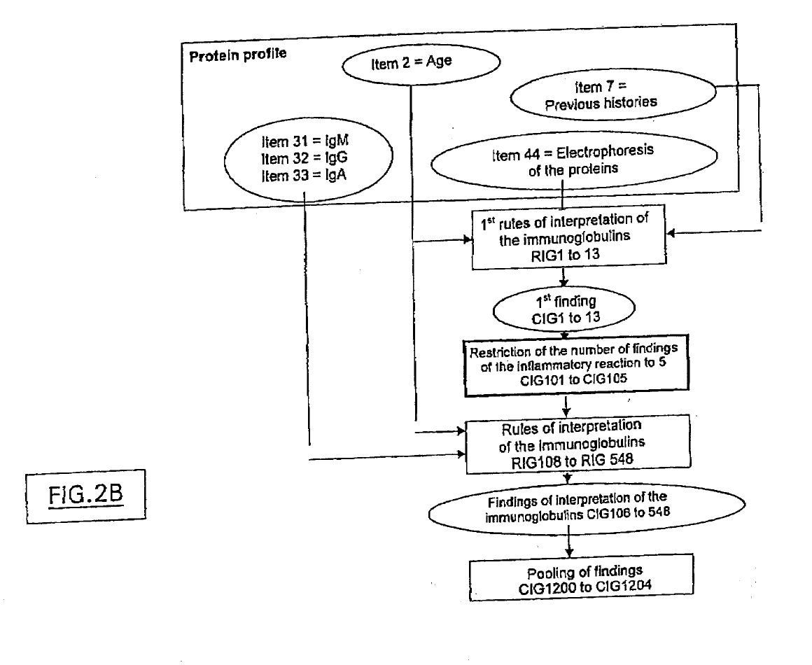

FIG. 2A illustrates an example of internal structure of the motor of an expert system according to the invention, relating more particularly to rules of the inflammatory reaction, and

FIG. 2B illustrates an example of internal structure of the motor of an expert system according to the invention, relating more particularly to rules of interpretation of immunoglobulin.

An expert system according to the invention of biological analysis according to the invention can in practice be implemented within a computer such as an office computer or a portable computer, and accessed locally or remotely. Its internal architecture, which can conform to current standards applying to expert systems, includes, with reference to FIG. 1, a module for collecting data determining profiles, respectively biological (protein in particular) and genetic profiles, of a patient, a module for collecting personal information specific to the patient, rules of interpretation applied to a processing of the biological profile realised with a genetic interpretation, and an editing of findings that can be used by a practitioner user.

The set of rules contained in this expert system according to the invention is organised into groups of rules each group corresponding to a group of specific analysis among several groups of analysis. For example, one may consider the group of rules of the inflammatory reaction or the group of rules interpreting immunoglobulins.

An embodiment of an expert system according to the invention will now be described, with reference to FIGS. 2A and 2B, being limited, for reasons of fullness of the description and clarity, only to the protein profile of a patient, it being understood that other specific biological profiles could be processed in an equivalent manner within the scope of the present invention.

In this expert system, a protein profile comprises optional items such as Item 43=ROP or Item 45=C4 and a set of obligatory items such as the following items:

| | |
|---|---|
| Item 2 = Age | Item 39 = TRF |
| Item 3 = Sex | 39.1 = Normal |
| Item 35 = ORO | 39.2 = Increased (obligatorily > |
| 35.1 = Normal | 119, not below) |
| 35.2 = Increased | 39.3 = Reduced |
| 35.3 = Much increased | Item 40 = ALB |
| 35.4 = Reduced | 40.1 = Normal or increased (> 89%) |
| Item 36 = HAPTO | 40.2 = Reduced (< 89%) |
| 36.1 = Normal | Item 41 = TRF/ALB |
| 36.2 = Increased | 41.1 = Normal |
| 36.3 = Much increased | 41.2 = Increased |
| 36.4 = Reduced | Item 42 = PAB |
| 36.5 = Much reduced | 42.1 = Normal or increased (> 84%) |
| 36.6 = Hapto < 10% | 42.2 = Reduced (<84%) |
| Item 37 = CRP | Item 44 Electrophoresis of |
| 37.1 = Normal < 33% | the proteins effected |
| 37.2 = Normal increased | NO |
| 37.3 = Increased | IgM |
| 37.4 = Much increased | IgG |
| 37.5 = Very much increased. . . | IgA |
| | Monoclonal peak not M, not G, not A |
| | Double monoclonal peak |
| | Absence of monoclonal protein |

The expert system according to the invention includes rules of the inflammatory reaction such as the following rules:

RINF1=35.1 +36.1 +37.1=CINF1

RINF2=35.1+36.1+37.2=CINF2

RINF100=35.4+36.5+37.5=CINF100

The findings associated with these rules of the inflammatory reaction are for example written up in the following way:

| | |
|---|---|
| CINF1 = | No inflammatory reaction as the proteins of the |
| | inflammation (CRP, Alpha-1-glycoprotein or orosomucoid, |
| | haptoglobin) are normal. |
| CINF2 = | The proteins of the inflammatory reaction are all in |
| | normal values. However, the level of CRP, although |
| | normal, may suggest the presence of microinflammations |
| | (to be taken into consideration in the assessment of the |
| | cardiovascular risk after formal elimination of any other |
| | potentially phlogogenic hearth). |
| . . . | |
| CINF100 = | The results lean towards an inflammatory process |
| | based solely on the strong increase in the CRP. This may |
| | be the start of an inflammatory process since the CRP, a |
| | protein of acute inflammation, increases more quickly than |
| | the other proteins of the inflammation. Such an induction |
| | leans towards an infectious and/or inflammatory hearth |
| | that is at the present time recent and very active, kept in |
| | an active state or in re-induction phase. The low level of |
| | haptoglobin is favourable for a hemolysis. Any |
| | haptoglobin result below 50% can be considered |
| | pathological. It may thus be of benefit to seek the cause |
| | of this hemolysis. Here, the reduced level of alpha-1- |
| | glycoprotein need not suggest a medicament treatment in |
| | the first place, but rather a protein leak, or a hepatocytic |
| | insufficiency. |

A table of the normal values makes possible the linking with numerical values of the items ORO, HPT, CRP of results such as:

| | | | MUCH | | MUCH |
|---|---|---|---|---|---|
| | NORMAL | INCREASED | INCREASED | REDUCED | REDUCED |
| ORO | 70 to 149% | 151 to 200% | >200% | <70% | — |
| HPT | 60 to 160% | 161 to 200% | >200% | 50 to 60% | <50% |

| | N1 | N2 | N3 | N4 | N5 |
|---|---|---|---|---|---|
| CRP | 0 to 66% | 67 to 200% | 201 to 666% | 667 to 2000% | >2000% |

| | | | | |
|---|---|---|---|---|
| Reminder | 100% = 3 mg | = 6 to 20 mg | = 21 to 60 mg | >60 mg |

| | | | MUCH | | MUCH |
|---|---|---|---|---|---|
| | NORMAL | INCREASED | INCREASED | REDUCED | REDUCED |
| ORO/ | 0.75 to 1.5 | >1.5 | — | <0.75 | — |
| HPT | | | | | |

There will now be described new rules of the inflammatory reaction corresponding to an interpretation of item 38 in relation to the findings for the three items 35, 36, 37.

RINF101=CINF1+138.1 [symbol] CINF101

RINF102=CINF1+138.2 [symbol] CINF102

RINF103=CINF1+138.3 [symbol] CINF103

RINF116=CINF7+138.1 [symbol] CINF101

RINF165=CINF31+138.2 [symbol] : IMPOSSIBLE

The set of findings corresponding to these rules includes for example:

| | |
|---|---|
| CINF101 = | No dissociation of the orosomucoid/haptoglobin pair |
| | which remains homogeneous. |
| CINF102 = | Dissociation between the alpha-1-glycoprotein and the |
| | haptoglobin. The dissociation leans towards a hemolysis. |
| | However, given the level of haptoglobin, the hemolysis is |
| | not necessarily to be regarded as pathological. |
| . . . | |

CINF107 = CINF 101
. . .

| | |
|---|---|
| CINF1400 = | This reduction, with orosomucoid/haptoglobin |
| | dissociation, may result from a desialyting activity affecting |
| | the orosomucoid such as taking dibasic medicaments |
| | modifying the antigenic structure of the protein (antibiotics, |
| | AINS, beta-blockers,. . . ) or resulting from a slight loss of |
| | protein through leakage of urinary, digestive, cutaneous |
| | origin as orosomucoid, which is a protein of low molecular |
| | weight, is very sensitive to the pathologies of leakage. |

The start of the protein profile thus presents itself as follows:

printing of the finding CINF1 to CINF 100

printing of the finding CINF101 to CINF240

The findings CINF1 to CINF100 are then linked with items 39, 40, 41, 42. Although there are actually only 60 different findings, this would lead to far too great a number of rules. It is thus proposed to make changes in these 60 findings in order to end up with a more limited number of findings, for example 6, with reference to FIG. 2A.

If 6 findings CINF301 to CINF306 are considered, linked with items 39 (TRF), 40 (ALB), 41 (TRF/ALB), 42 (PAB), 6×3×2×2×2, i.e. 144 complementary rules must be provided.

But the high TRF must be interpreted as a function of sex and age. Now, out of 144 rules, a high TRF is observed 48 times. 48×2 (sex) ×3 (age), i.e. 288 supplementary rules must therefore be provided. In addition, within the sex and age, there is the criterion of whether a woman is menopausal or not, which leads to 12×6, i.e. 72 complementary rules.

The total number of rules is thus 144+288+72, i.e. 504 rules. However, 96 rules actually prove to be impossible. There are thus 408 possible different rules.

The 6 resultant findings are:

1) CINF301: no, or very slight, inflammatory reaction

2) CINF302: inflammatory reaction based solely on the increase in CRP

3) CINF303: inflammatory reaction with increase in only one protein of the chronic reaction

4) CINF304: clear inflammatory reaction with normal CRP

5) CINF305: clear inflammatory reaction with increased CRP

6) CINF306: reduction in the proteins of the chronic inflammation

The 408 possible different rules include:

RINF307=CINF301+139.1+140.1+141.1+142.1=CINF307

RINF738=CINF306+139.3+140.2+141.2+142.2=CINF738

The interpretation of the Ig (immunoglobulins) in the protein profile will now be considered. The items concerned are 131 (IgM), 132 (IgG) and 133 (IgA). The interpretation is different depending on whether there is or not a monoclonal protein. Now, the presence of a monoclonal protein is not visible in the protein profile but in another analysis which is electrophoresis of the proteins.

Now, this electrophoresis is not always requested together with a profile. Moreover, if it is carried out, a monoclonal protein is found only rarely. When a monoclonal protein is found, the interpretation stops there, and this finding is not linked with an inflammatory reaction. Thus, the interpretation of the Ig starts with the processing of item 44 “Electrophoresis of the proteins”.

The reply may be:

yes with presence of a monoclonal protein (1^{st} finding),

yes with absence of monoclonal protein (new 1^{st} finding),

no (new 1^{st} finding).

Any individual can present Ig levels outside the standard values without this being pathological. What is pathological is the variation in this level of Ig over two taking, hence the processing of item 7 “previous histories”.

If the reply is no, this means a 2^{nd} finding of a general order before the actual processing of the Ig.

Items 31, 32, 33 must then be linked with the inflammatory reaction. The findings of the interpretation of the immunoglobulins have been reduced to 5 according to a method similar to that adopted for the inflammatory reactions:

no inflammatory reaction

inflammatory reaction

inflammatory reaction due solely to CRP

inflammatory reaction present (1, 2 or 3 proteins)

reduction of the proteins of the inflammatory reaction.

The 3^{rd} finding will thus be chosen from among the following rules:

131×132×133×C5

5×3×5×5×=375 new rules.

The first finding can be established in the following way:

RIG1=144.2.1+12.1=CIG1

RIG13=17.2=CIG13

| | |
|---|---|
| CIG1 = | The electropho resis and the immunoelectrophoresis |
| | revealed a monoclonal IgM. Given the patient's age, one |
| | must think first of a sub-acute or chronic severe infection |
| | or viral or bacterial origin. This suggests an associated |
| | immunodeficiency. |
| . . . | |
| CIG13 = | The values of the Ig reflect all of the defences acquired |
| | during life as a function of encounters with the different |
| | pathogens. At adult age, in a healthy person, this level does |
| | not vary much. It is thus perfectly possible that a level |
| | outside the normal values has no pathological connotation, |
| | but be a perfectly physiological level for the patient. What |
| | is interesting, is the assessment of the variation over two |
| | samples several months apart. Not having any prior history |
| | for this patient, the different etiologies proposed enjoy only |
| | indicative status, as the interpretation must be carried out |
| | above all in relation to the clinical context. |

As indicated above, the 60 different findings of the inflammatory reaction are reduced to 5, so that the following findings are determined:

No Inflammatory Reaction

CINF1 or CINF2 or CINF3 or CINF17 or CINF21 or CINF22 or

CINF23 or CINF6 or CINF16 or CINF18 or CINF26 or CINF76 or

CINF77 or CINF78 =CIG101

Slight Inflammatory Reaction

CINF7 or CINF8 or CINF31 . . . or CINF83=CIG102

Inflammatory Reaction Due Solely to CRP

CINF4 or CINF5 or CINF19 . . . or CINF80=CIG103

Inflammatory Reaction Present (Due to One or More Proteins)

CINF9 or CINF10 or . . . or CINF34 . . . or CINF90=CIG104

Reduction in Proteins

CINF91 or CINF92 or CINF93 or CINF94 or CINF95 or CINF96 or

CINF97 or CINF98 or CINF99 or CINF100=CIG105

The 375 rules for the 3^{rd} finding are then established, such as by way of example:

RIG106=CIG101+131.1+132.1+133.1=CIG106

RIG548=CIG105+131.5+132.3+133.5=CIG548

Complementary rules as a function of age are added to take account of the situations where each time there will be an inflammatory reaction (CIG104) without any increase in the Ig, or with a reduction in the IgM. In order to create these complementary rules, a pooling of certain of the CIG××× findings mentioned above is carried out in order to end up with 5 findings CIG1200, CIG1201, CIG1202, CIG1203, CIG1204 which are used in the establishment of these complementary rules.

An embodiment of the method of processing data according to the invention will now be described, for a combined interpretation of the genetic profile and cardiovascular risk.

Firstly, a non-exhaustive list of genes that can be interpreted within the scope of the expert system of biological analysis according to the invention is provided in table I below. For each gene, a + symbol in a column indicates that this gene plays a part in the characteristic corresponding to this column, and conversely a − symbol in another column indicates that the same gene does not play a part in the characteristic corresponding to this other column. Thus, by way of example, the gene CYP1A1 plays a part in the case of smoker and as regards nutrigenetics, but not as regards pharmacogenetics, immunogenetics and for oxidative stress. Thus, each + symbol in this table corresponds to links and rules which must be written and integrated into the expert system.

There follow, by way of non-limitative example, extracts of biological interpretation supplied by an expert system according to the invention, regarding cardiovascular risk:

“in the light of the biological results, there is no atherogenic risk.

The other risk factors must therefore be explored, as nearly 20% of patients who have cardiovascular problems present a normal or sub-normal biology.”

[. . . ]

“Hyperhomocystinemia caused by congenital deficiency of the enzymes involved in its biosynthesis is much more rare. For example, cystathionine-beta-synthase deficiency is estimated at 1/20000 subjects who, in addition to cardiovascular risk, also have mental backwardness, and a dislocation of the crystalline lens, osseous deformations. On the other hand, 5-10 methylinetetrahydrofolate reductase deficiency is more frequent, being estimated at 5% of the general population, and is the major cause of genetic predisposition to moderate hyperhomocystinemia. These patients often present cardiovascular disorders in the first years of life [. . . ]”

This constitutes an indication for conducting genetic tests in order to know whether the increase in homocysteine is genetic in origin or not.

“Although the E2 allele seems to play a part in type III hyperlipoproteinemias, the E4 allele is also more involved in cardiovascular diseases. The E2/E4 genotype, although infrequent, thus substantially increases the risks of cardiovascular problems. Generally speaking, the average cholesterolemia of E4/E3 subjects is greater than that of E3/E3 subjects, which is itself greater than that of E3/E2 subjects. In the same way, the average concentration of LDL cholesterol in E4/E3 subjects is greater than that of E3/E3 subjects, which is itself greater than that of E2/E2 subjects. On the other hand, the triglycerides are significantly higher in E2/E2; E3/E2, E4/E2 subjects than in E3/E3; E4/E3 subjects.”

This finding reflects a direct relationship between interpretation of a genetic profile and interpretation of a biological profile (cholesterol, triglycerides).

There is presented below an example of a finding reflecting a direct relationship between genetics and diet:

“Subjects carrying the E4 allele are more sensitive to hypolipemic and hypocholesterolemic diets. In the same subjects, the return to a diet rich in fats, in particular in saturated fatty acids, leads to a greater increase in plasmatic cholesterol.”

The expert system according to the invention can also take account, in the findings supplied to the user, of a direct relationship between the interpretation of the genetic profile and data relating to the medicament treatment that are obtained from personal information specific to the patient, as illustrated by the finding presented below:

“Subjects carrying the E2 allele and affected by hyperlipoproteinemia of lib type respond well to treatment by gemfibrozil and by statins (simvastatin and lovastatin). Among subjects affected by hyperlipoproteinemia of lia type, carriers of the E2 or E3 allele respond well to treatment by statins. Subjects carrying the E4 allele would on the other hand respond less well to hypolipidemic medicamentous treatments, with the exception, perhaps, of probucol.”

The invention is, of course, not limited to the examples which have just been described and numerous modifications can be made to these examples without exceeding the scope of the invention. In particular, provision can be made for complete automation of the operations for determining the biological profile and the genetic profile of a patient, and the combined treatment of these profiles. Moreover, it will easily be understood that an expert system of biological analysis according to the invention can also be coupled with databases and knowledge bases. In addition, within the framework of the present invention, the biological profile not only includes several families of determinations and biological analysis which are henceforth well established such as protein profiling or lymphocyte typing, but also other profiles in the process of being developed or which will be proposed in the future. In the same way, the expert system according to the invention is intended to take account of increasingly complex genetic profiles as scientific and technological advances occur in this field.

TABLE I

| | Predisposition | | | | | |
|---|---|---|---|---|---|---|
| Genes | to disease | Pharmacogenetic | Immunogenetic | Smoker | Stress O. | Nutrigenetic |
| Phase I of bio- | | | | | | |
| transformation | | | | | | |
| CYP1A1 | + | − | − | + | − | + |
| CYP1A2 | + | + | − | + | − | − |
| CYP2A6 | + | + | − | + | − | − |
| CYP3A4 | + | + | − | − | − | + |
| CYP2B6 | + | + | − | − | − | + |
| CYP1B1 | + | + | − | − | − | + |
| CYP2D6 | + | + | − | − | − | − |
| CYP2E1 | + | − | − | − | − | + |
| CYP2C19 | + | + | − | − | − | − |
| CYP2C9 | − | + | − | − | − | − |
| MEH | + | − | − | + | − | − |
| ALDH | + | − | − | + | − | − |
| ADH2 | + | − | − | + | − | − |
| Phase II of bio- | | | | | | |
| transformation | | | | | | |
| GSTM1 | + | + | + | + | + | + |
| GSTM3 | + | − | − | − | + | + |
| GSTT1 | + | − | − | + | + | + |
| GSTP1 | + | + | − | − | + | + |
| NAT2 | + | + | + | + | − | + |
| NAT1 | + | + | + | + | − | + |
| Trigger genes | | | | | | |
| Osteoporosis | | | | | | |
| Vit D3 | + | − | − | − | − | − |
| Col1A1 | + | − | − | − | − | − |
| ER | + | − | − | − | − | − |
| CTR | + | − | − | − | − | − |
| AIDS | | | | | | |
| CCR5 | + | − | − | − | − | − |
| SDF1 | + | − | − | − | − | − |
| CCR2 | + | − | − | − | − | − |
| CXCR4 | + | − | − | − | − | − |
| Breast cancer | | | | | | |
| BRCA1 | + | − | − | − | − | − |
| BRCA2 | + | − | − | − | − | − |
| Prostate cancer | | | | | | |
| AR | + | − | − | − | − | − |
| Hereditary | | | | | | |
| trorabophilia | | | | | | |
| Factor V | + | − | − | − | − | − |
| Hemochromatosis | | | | | | |
| HFE | + | − | − | − | − | − |
| Bronchial and | | | | | | |
| allergic asthma | | | | | | |
| CC16 | + | − | − | − | − | − |
| AAT-locus | + | − | − | + | − | − |
| HNMT | + | − | − | + | − | − |
| PAFAH | + | − | − | + | − | − |
| AACT | + | − | − | + | − | − |
| Primary Hyperchol- | | | | | | |
| esteremia | | | | | | |
| LDLR | + | − | − | − | − | − |
| APOB | + | − | − | − | − | − |
| Cardiovascular risk | | | | | | |
| MTHFR | + | − | − | − | − | − |
| ACE | + | − | − | − | − | − |
| Efflux genes | | | | | | |
| MDR1 | − | + | − | − | − | − |
| MDR3 | − | + | − | − | − | − |
| LRP | − | + | − | − | − | − |
| MRP1 | − | + | − | − | − | − |
| Other metabolizing | | | | | | |
| genes | | | | | | |
| NQO1 | + | − | − | − | + | − |
| Cytokine genes | | | | | | |
| IL-1a | + | − | + | − | − | − |
| IL-1b | + | − | + | − | − | − |
| ILRN | + | − | + | − | − | − |
| IL-2 | + | − | + | − | − | − |
| IL-4 | + | − | + | − | − | − |
| IL-6 | + | − | + | − | − | − |
| IL-9 | + | − | + | − | − | − |

1. Method of processing biological analysis data, comprising processing of a set of data relating to the biological profile of a human or animal subject, in the form of biological items to which a set of rules supplying findings in the form of statements in natural language are applied, characterized in that the said set of rules includes a plurality of groups of rules each associated with a group of analysis among a plurality of groups of analysis constituting the biological profile, and in that it also comprises at least one operation pooling a set of findings that have resulted from a group of rules, so as to limit the number of findings which are entered a following group of rules.

2. Method according to claim 1, characterized in that it also comprises processing of genetic data relating to this patient in the form of a set of genetic items associated with a set of genes studied in this patient, this treatment comprising the application of rules of genetic interpretation applied at the same time to biological items and to genetic items.

3. Expert system of biological analysis applying the method of processing data according to anyone of the previous claims, said system comprising:
means of collecting data resulting from biological measurements carried out on a human or animal subject and defining a biological profile, and personal data relating to the said human or animal subject, these data being represented in the form of a set of items,
a motor comprising several groups of pre-established rules, each of the said rules relating to certain items selected from among the set of items and/or certain findings of rules applied before the said rule, and
means of issuing findings in reply to the application of the said rules,
characterised in that it further comprises means of pooling a set of findings that have resulted from a set of rules associated to an analysis group among a plurality of analysis groups constituting the biological profile, so as to limit the number of findings which are entered a following group of rules.

4. Expert system according to claim 3, characterised in that it further comprises means of collecting data relating to the genetic profile of the said human or animal subject in the form of genetic items, and in that the motor also comprises rules for the interpretation of the said genetic items.

5. Expert system according to claim 3 or 4, characterised in that it is integrated into an automated system including operations determining the biological profile and the genetic profile of patients.

6. Expert system according to one of claims 3 to 5, characterised in that it is coupled with a knowledge base.

7. Expert system according to one of claims 3 to 6, wherein the biological profile includes a protein profile.

8. Expert system according to one of claims 3 to 7, wherein the biological profile includes a lymphocyte typing.
